(19) United States
(12) Patent Application Publication
(10) Pub. No.: US 2025/0213412 A1
(43) Pub. Date: Jul. 3, 2025

(54) BODY PART SUPPORT

(71) Applicant: KING'S COLLEGE LONDON, London, Greater London (GB)

(72) Inventors: Simon Richardson, London (GB); Frederic Dick, London (GB); David Carmichael, London (GB); Martina Callaghan, London (GB)

(21) Appl. No.: 18/853,081

(22) PCT Filed: Apr. 6, 2023
(86) PCT No.: PCT/GB2023/050932

§ 371 Date: Sep. 30, 2024

(30) Foreign Application Priority Data
Apr. 7, 2022 (GB) 2205139.5

(51) Int. Cl. A61G 13/12; A61B 5/00; A61B 5/055; G01R 33/34

(52) U.S. Cl. CPC A61G 13/1265; A61B 5/0042; A61B 5/055; A61G 13/121; A61G 13/1285; G01R 33/34084; A61G 2203/34; A61G 2210/50

(57) ABSTRACT

Aspects and embodiments relate to a body part support, a device comprising a body part support and a method of supporting a body part. More particularly, aspects and embodiments may relate to a medical imaging body part support. The imaging support of various aspects and embodiments comprises an inflatable bladder. Aspects and embodiments provide a body part support comprising: a frame and an inflatable bladder. The frame comprises a cover portion and a base portion. The cover and base portion are reversibly moveable with respect to each other between an open position and a closed position. In the open position the body part is locatable within the base portion. In the closed position the cover portion and base portion are securable together to encircle at least a portion of the body part locatable within the base portion. The inflatable bladder is mountable on the frame, located such that inflation of the inflatable bladder causes a reduction of a gap formed between the interior of the frame and encircled portion of the body part locatable within the base portion. An arrangement in accordance with described aspects mitigates body part movement within the support.

### FIELD OF THE INVENTION

Aspects and embodiments relate to a body part support, a device comprising a body part support and a method of supporting a body part. More particularly, aspects and embodiments may relate to a body part support for medical imaging. The imaging support of various aspects and embodiments comprises an inflatable bladder.

### BACKGROUND

Various medical imaging techniques are known. Each imaging technique provides a mechanism by which data from at least one aspect of a subject body can be captured or “imaged” to provide information which may be of use in relation to analysis of structure or function of the subject body, for example, in support of research, medical study and/or medical intervention. Such imaging may also provide information of relevance to the study, diagnosis and/or treatment of disease.

Some primary known medical imaging techniques include: various radiography applications such as, magnetic resonance imaging (MRI); computerised tomography (CT) and X-rays; and ultrasound applications. Radiography uses X-rays, gamma rays, or similar ionising and non-ionising radiation to allow creation of an image of the interior of a subject body. MRI uses strong magnetic fields, magnetic field gradients, and radio frequency electromagnetic waves to collect data relating to create images the interior of a subject body, that data can be used to create images representative of the interior of a subject body. CT images are created using information gathered about the interior of a subject body using X-rays, gamma rays, or similar and ultrasound uses sound waves to create an image of the interior of a subject body. One or more of the various known medical imaging techniques can be selected based upon the information which is required about the interior of a subject body. For example, MRI techniques can provide good contrast in images of soft tissues, as may be required in relation to images of a brain; X-ray techniques may provide good images of dense material within the interior of a subject, including bones and teeth. Ultrasound techniques may be selected in relation to foetal imaging, to mitigate any risk associated with exposure of the interior of a subject body to ionising radiation.

It will be appreciated that obtaining a clear and accurate image of the interior of a part of a subject body may be impeded if the body part, or interior of the body part, is subject to significant movement during image capture. Such movement can cause detrimental image artefacts and result in a reduction in resulting image quality.

Although various mechanisms to address issues caused by movement of a part of a subject body during image capture may be known, it is desired to provide an alternative mechanism by which image quality in medical imaging of a body part of a subject may be improved.

### SUMMARY

A first aspect provides a body part support comprising: a frame and an inflatable bladder; wherein: the frame comprises a cover portion and a base portion, the cover and base portion being reversibly moveable with respect to each other between: an open position in which the body part is locatable within the base portion; and a closed position in which the cover portion and base portion are securable together to encircle an exterior surface of at least a portion of the body part locatable within the base portion; the inflatable bladder being mountable on the frame, located such that inflation of the inflatable bladder causes a reduction of a gap formed between the interior of the frame and encircled portion of the exterior surface of the body part locatable within the base portion.

The first aspect recognises that imaging of a part of a subject body may be assisted if that part of a subject body is substantially immobile during an image capture process. In particular, the first aspect recognises that by supporting a body part in a manner which makes it substantially immobile, it is possible to mitigate imaging issues resulting from motion, including unwanted artefacts and an overall reduction in image quality. Furthermore, imaging of a body part which is substantially immobile aids imaging reliability and reproducibility.

Various arrangements and methods to support a body part may be envisaged, including use of restraints, foam pads or wedges, support pillows, taping, bespoke mouldings and/or casts, to restrict, restrain, support or otherwise hold a body part of a subject in position. Such arrangements can cause discomfort to a subject being imaged, causing the subject to move in order to try and find a more comfortable position, thereby not achieving the desired result. Some such arrangements may only restrict movement about a single axis, rather than several axes, again limiting the extent to which a body part may be held substantially immobile during an image capture process.

The usefulness of such arrangements may be restricted to particular medical imaging techniques, depending on their nature. By way of example, it will be appreciated that particular materials used for restraints or supports may be unsuitable for use in radiographical applications. Similarly, material choice of such arrangements may render them unsuitable for a support or restraint which is to be located within an MRI scanner. Furthermore, medical imaging devices may comprise delicate or expensive components which may be detrimentally impacted by use of some envisaged support or restraint arrangements.

The first aspect seeks to mitigate some of the issues outlined above. Aspects relate to imaging subject supports. Aspects may provide a restraint or support for supporting a body part or body parts of an imaging subject. The body part may be supported as described, for example, in order to perform imaging of the body part, but it will be appreciated that aspects may provide support or restraint to a body part during a medical examination or treatment.

The first aspect recognises that it can be problematic to insert or place a body part within a body part support whilst also providing appropriate support or restraint to that body part. A substantially open support, for example, allows for ease of insertion of a body part, but may not provide sufficient restraint or restriction to movement of the body part. Provision of a support which provides an open configuration in which movement of a body part is substantially unimpeded in at least some axes allows for ease of insertion of the body part into the support. The first aspect recognises that providing a closed configuration in which a frame of the support encircles at least a portion of the body part allows for improved restriction of the body part whilst in the closed support device. The annular, encircling, closed portion of the frame can provide a structure against which a restraining force can be exerted, thereby allowing for restriction of movement of the body part locatable within the support.

A first aspect may provide a body part support. That body part may comprise, for example, a head, arm, leg, or part of a head, arm, or leg. Aspects may provide a head support, arm support, a hand support, wrist support, elbow support, leg support, foot support, ankle support, knee support or similar. The first aspect may comprise: a frame. The frame may be formed from resilient material, selected to have an appropriate rigidity to resist expansion or deformation of the frame on exertion of expected forces from within the frame by any restraining elements in combination with a body part located within the body part support. The body part support may comprise at least one inflatable bladder. The inflatable bladder may comprise at least one sack or bag, into which a fluid may be placed to increase the effective volume of the inflatable bladder. The inflatable bladder may comprise two or more inflatable bladders. The inflatable bladder may be formed of more than one individually inflatable compartment or cell. The effective volume of the inflatable bladder may be adjustable. The adjustment may occur as a result of insertion of a suitable fluid into the bladder. The fluid insertable into the bladder may comprise a gas. The fluid insertable into the bladder may comprise air. The frame of the body part support may comprise two main sections. Those main sections may comprise: a cover portion and a base portion. The cover and base portion may be moveable with respect to each other between an open position and a closed position. When in the open position the body part to be supported can be easily located within the base portion in a manner which is unimpeded by the frame. When in the closed position, the cover portion and base portion may be secured together. When in the closed position, the cover and base portion may together encircle at least a portion of the body part locatable within the base portion. In the closed position the frame may thus form a continuous annulus around at least a portion of a body part to be supported. The inflatable bladder may be affixed to, or removably mountable on, the frame. Whether affixed or mountable, the inflatable bladder may be located, in use, such that inflation of the inflatable bladder by increasing its effective volume, may cause a reduction of a gap formed between the interior of the frame and an exterior surface of the body part. That reduction in gap may occur around the encircled portion of the body part locatable within the base portion.

The frame may be formed from a material having a rigidity sufficient to resist deformation when the bladder is fully inflated and the body part locatable within the base portion is in position.

Accordingly, the frame is formed from a material which has sufficient rigidity, for example, when shaped and dimensioned to accommodate a particular body part, the rigidity being selected to prevent translated force which may occur when the inflatable bladder inflates and pushes against the body part, in turn pushing against the interior surface of the frame, from affecting anything surrounding the outer surface of the frame. As a result, any imaging or surgical equipment which may surround the body part support can be substantially insulated against the forces being applied to the body part within the frame to support and/or restrain the body part. Such a feature may be helpful, for example, if the support is to be used as a body part imaging support and in which the support is to be located within a delicate or force sensitive imaging device.

The external surface or surfaces of the body part support may be shaped to substantially conform to an interior surface of an imaging device within which the body part support is locatable.

Accordingly, in some implementations, the external surfaces of the frame may be moulded or formed, for example, by additive manufacture, 3-D printing or similar, to be a close or friction fit within, for example, an imaging device. In some cases, the body part support may be an integral part, or integratable with, an imaging device. By matching the outer surface of the body part support to main contours of an interior surface of a device within which it is intended for use, it may be possible to evenly distribute any potential forces which are translated to the outer surface of the frame as part of support or restraint of a body part within the support. In other words, by shaping the external surface of the support to substantially conform to the interior of a device within which it can be placed, it is possible to ensure that an even pressure is exerted on the interior surface of the imaging device and thus to mitigate the chance of damage to the imaging device as part of supporting or restraining a body part.

The cover portion and base portion may be hingeably connected. The cover portion and base portion may be connected via a flexible coupling. The flexible coupling may comprise a moveable joint or joints(s) comprising a mechanical linkage. Accordingly, alignment of the cover portion and base portion of the frame may be maintained, whilst allowing for ease of movement between open and closed configurations. Furthermore, connecting the cover and base portion in a restricted, but openable manner can support ease of insertion and removal of a body part into the support, while maintaining alignment of the frame.

The body part support may comprise one or more cooperating elements configured to lock the cover portion and base portion in the closed position. Accordingly, the locking mechanism may provide a structural link between the cover portion and base portion. The cooperating portions may be located at the region of the frame in the closed position which encircles the portion of the body part locatable within the base portion. Accordingly, the locking mechanism may assist in providing resistance from the frame to forces which are translated to the frame as part of support or restraint of a body part within the support.

The inflatable bladder may be formed from a substantially inelastic material. The inflatable bladder may be formed from an elastic material. The inflatable bladder may be formed from a supple, pliable material. Accordingly, when uninflated the bladder may occupy a small volume within the frame of the support. As fluid, for example, gas, inflates the bladder, the bladder may increase in volume and occupy a greater volume within the frame of the support. Accordingly, the volume occupied by the inflatable bladder within the frame may be adjustable. Forming the inflatable from an appropriate material can help to ensure smooth and comfortable inflation and deflation is experienced by a subject having their body part supported. The inflatable bladder(s) may be formed from a material and sized such that full inflation of the bladder remains within the elastic limit of the bladder. That is to say, the material and size characteristics of the inflatable bladder may be selected such that normal use of the bladder can occur with a level of material stretching which does not cause a permanent alteration of size or shape of the inflatable bladder. Appropriate selection of material can help to ensure that an inflation level implemented in relation to an inflatable bladder in the body part support can be reproducible.

One or more of the inflatable bladder(s) may inflate to form: a pillow, wedge, torus, cuboid, organically curved 3D shape (for example, polygonal pillow) or one or more combinations thereof. Accordingly, the bladder(s) can be shaped to exert a directional force upon a body part locatable within the support. The inflatable bladder(s) may be shaped to exert a force upon the body part and move it towards an interior surface of the frame opposing the surface upon which the inflatable bladder is mountable.

The inflatable bladder(s) may comprise an opening configured to receive fluid from a fluid source. The opening may be configured to allow exit of gas from the inflatable bladder. Each inflatable bladder may be provided with one or more opening for ingress and/or egress of fluid from the respective inflatable bladder. Accordingly, the inflatable bladder may comprise a single opening which functions as both an inlet and outlet for a fluid entering or exiting the bladder. Some arrangements may provide a separate inlet and outlet opening. Some arrangements may provide a bladder with more than one inlet and/or outlet. Those inlets and/or outlets may allow for independent inflation or deflation of one or more independent section(s) of the inflatable bladder. Those inlets and/or outlets may allow for independent inflation or deflation of one or more inflatable bladder where more than one inflatable bladder is provided.

The body part support may comprise one or more deformable pad, mountable on the frame. The deformable pad may be provided on an interior surface of the frame. The deformable pad may be located on the frame facing the inflatable bladder. Accordingly, the deformable pad may, in some implementations, be located on an interior surface of the frame to exert an opposing force upon the body part to the inflated bladder. Such an arrangement can aid body part comfort and also help to ensure the body part is restrained by an even exertion of opposing force to that exerted by an inflated bladder.

The body part support may comprise a pair of inflatable bladders positioned opposite or facing one another on the interior of the frame. The pair of inflatable bladders may be positioned on an axis orthogonal to an axis joining the inflatable bladder to a point on the interior of the frame opposite the inflatable bladder. Inflation of the pair of bladders may cause a reduction of a gap formed between the pair of bladders and the body part locatable within the frame. The pair of inflatable bladders may comprise a pair of bladders having a substantially identical shape when inflated. The pair of inflatable bladders may comprise a pair of bladders having a substantially identical mirrored shape when inflated. Accordingly, provision of a pair of inflatable bladders may provide restriction and support of body part movement in a different axis to that which is restricted by the provision of a first inflatable bladder mountable on the frame.

The pair of inflatable bladders may be formed from a substantially inelastic material. The pair of inflatable bladders may be formed from an elastic material. The pair of inflatable bladders may be formed from a supple, pliable material. Accordingly, when uninflated the pair of inflatable bladders may occupy a small volume within the frame of the support. As fluid, for example, gas, inflates the pair of inflatable bladders, pair of inflatable bladders may increase in volume and occupy a greater volume within the frame of the support. Accordingly, the volume occupied by the pair of inflatable bladders within the frame may be adjustable. Forming the inflatable from an appropriate material can help to ensure smooth and comfortable inflation and deflation is experienced by a subject having their body part supported. The pair of inflatable bladders may be formed from a material and sized such that full inflation of the bladder remains within the elastic limit of the bladders. That is to say, the material and size characteristics of the pair of inflatable bladders may be selected such that normal use of the pair of inflatable bladders can occur with a level of material stretching which does not cause a permanent alteration of size or shape of the pair of inflatable bladders. Appropriate selection of material can help to ensure that an inflation level implemented in relation to the pair of inflatable bladders in the body part support can be reproducible.

Each of the pair of bladders may be independently inflatable. As described above in relation to a single inflatable bladder, each of the pair of inflatable bladders may comprises an opening configured to receive fluid, for example, gas, from a fluid source. Each opening may be configured to allow exit of fluid from the respective inflatable bladder of the pair of inflatable bladders.

A least one of the inflatable bladders mountable on the frame, for example the inflatable bladder or one or more of the pair of inflatable bladders, may comprise one or more cells in fluid communication, the cells being dimensioned to give the bladder a predetermined shape when inflated. Accordingly, one or more of the bladders can be shaped to accommodate a particular feature of a body part to be supported. Such a feature may, for example, comprise an ear, a curve of a skull, a bent joint or similar. The shaping of one or more bladder may aid subject comfort.

At least one of the inflatable bladders mountable on the frame may be coupleable to a fluid source. Accordingly, inflation of the one or more bladders may be achieved by provision of fluid to the bladder from the fluid source. The fluid may comprise a gas.

At least one of the inflatable bladders and/or one or more deformable pads may be positionable on the frame such that, when the frame is in a closed position, a stretching force is exertable upon a surface of the body part locatable within the support. Non-invasively securing a body part mechanically occurs by gripping skin on the surface of that body part. Skin is typically not rigidly attached to the musculoskeletal system and therefore a body part may be able to move, rotate etc in relation to the skin covering the body part. By way of example, the skin on the top of the head can move freely over ˜10 mm in any direction independent of the skull and musculature underneath. Gently stretching the skin can help to minimise motion of the skin relative to substructures and therefore minimise motion of a body part overall relative to an imaging device. According to some arrangements, the gentle stretching of the skin can be achieved by appropriate placement of the bladder(s) and pad(s). The bladders and pads may be shaped to provide a large contact surface in key areas of a body part (for example, the top and sides of the head) and shaping and locating opposing pads or bladders such that tension is applied to body part skin upon closure of an open frame. The gentle stretching of the skin in one or more key locations in relation to a body part can assist to lock a body part in position. The opposing inflatables and pads may provide a stable and comfortable system that reduces motion markedly, and accounting for the non-rigid attachment of skin to a body part by configuring those elements of the apparatus to stretch the skin can provide additional restriction of movement to a restrained body part.

The body part support may comprise a fluid flow control manifold, coupleable to: (i) one or more inflatable bladder opening configured to receive fluid from a fluid source and (ii) a fluid source. The fluid may comprise a gas. The manifold may comprise one or more valves reconfigurable to allow or prevent gas flow from a gas source to one or more inflatable bladder. The manifold may comprise one or more valves reconfigurable to allow or prevent gas flow from a gas source to each inflatable bladder. Accordingly, provision of an appropriate manifold may provide a mechanism for reliable and reproducible inflation and/or deflation of one or more inflatable bladder mountable on the support frame. Such a manifold may allow for adjustable support of a body part within the support.

The manifold may comprise a flowmeter. The manifold may comprise a fluid pressure meter. Accordingly, fluid flow to, or fluid pressure in, one or more inflatable bladder(s) may be monitored. According to some a implementations, a digital flowmeter or fluid pressure meter may be provided. The body part support may comprise a pressure sensor associable with one or more inflatable bladder. A digital or other visible readout may be visible to an operator. Provision of a flowmeter or pressure meter can facilitate repetition of the same inflation level in each inflatable bladder. Provision of a fluid pressure meter may, for example, allow an operator to spot any fluid leakage from the system or from a particular inflatable bladder.

The manifold may be configured to allow independent inflation of each inflatable bladder. The manifold may be configured to allow independent deflation of each inflatable bladder. Accordingly, support offered to the body part may be adapted to suit a particular restraint or support requirement. Such independent adjustment may also allow for restraint to be adjusted for the purposes of subject comfort.

The manifold may be configured to allow simultaneous deflation of all inflatable bladders. Accordingly, quick release of a body part from the support may be facilitated.

It may be possible to monitor pressure exerted by one or more inflatable bladder upon a body part. Inflation of bladder(s) to a predetermined pressure may ensure patient comfort and optimal function of the body part support. In some arrangements it may be possible to mitigate potential discomfort caused to a patient by monitoring or controlling pressure exerted by an inflatable bladder on a body part. In some arrangements it may be possible to select or restrict pressure exerted upon a body part in dependence upon the body part being supported. The pressure sensor may be coupled to the manifold. If a pressure reading exceeds a predetermined threshold, the manifold may be triggered to cause deflation of one or more inflatable bladder.

The body part support may comprise at least one deformable pad slidably mountable on the frame. Accordingly, movement of the slidably mounted deformable pad may allow fine adjustment of support or restraint of the body part within the frame.

The inflatable bladders and deformable pads may be dimensioned such that they are mountable at positions on the frame in which there is a space between adjacent bladders and pads. The inflatable bladders and deformable pads may be removably mounted at positions on the frame in which there is a space between adjacent bladders and pads. Accordingly, airflow around the body part may be maintained whilst it is supported and restrained, thereby aiding cooling and subject comfort.

The frame may comprise one or more cooling passages, channels or conduits provided on or in an interior, or exterior surface. The cooling passages, channels or conduits may form part of a cooling circuit. A cooling fluid may be passed along or through the cooling passages, channels or conduits. In one implementation, the cooling channels may comprise open topped trenches provided in an exterior surface of the frame. Accordingly, the open channel or trench may facilitate easy cleaning. A cooling ‘tube’ may be formed if the body part support frame is placed within an imaging device. An interior surface of the imaging device may “cover over” the open channel. The resulting fluid pathways may be provided with cooling fluid, for example, air. A cooling fluid may be coupled to the cooling passages, channels or conduits. The cooling passages, channels or conduits may be located on the support frame in one or more positions to cool a body part locatable within the support.

The body part support may comprise a head support. The body part support may comprise an MRI imaging head support. Accordingly, aspects described may be of particular use in relation to MRI imaging. MRI imaging can provide significant detailed information regarding the interior, particularly soft tissue, of a body part of a subject. MRI image capture typically requires a subject, or at least a body part of a subject, to remain substantially motionless for an extended period of time to support image capture. Whilst MRI imaging allows for collection of information which can have significant clinical and research value, use of MRI imaging can be perceived as less comfortable by subjects, due to the typically long and loud image capture process during which a subject may be located in a narrow, restrictive and confined space, for example, an MRI scanner. During an extended MRI image capture process, a subject may feel anxious, vulnerable and/or experience discomfort which can lead to unexpected subject movement within the bore.

In the case that the body part support comprises a head support, one or more inflatable bladder may comprise an audio delivery device. In particular, a bladder locatable around an ear may be provided with an audio delivery element, for example, a headphone or earbud. Accordingly, sound delivery apparatus may be included within an over ear inflatable, therefore allowing for immobilisation of a subject together with an ability to provide a subject with audio which may aid a patient in feeling comfortable during a procedure.

Accordingly, aspects described may be of particular use in relation to imaging of the head. Such imaging may include, for example, imaging of the brain. Such imaging may include MRI imaging of the brain. MRI imaging of the human brain requires a subject to remain still for an extended period. Whilst it might be expected that a subject will be able to control and limit movement of their head, motion of the head during imaging often occurs. During MRI brain imagining, a subject may be required to lie supine and remain substantially still for an extended period within a bore of an MRI scanner. The enclosed restrictive space, loud, potentially hot environment and extended image capture process can lead to additional subject movement. Aspects provide an arrangement in which a head can be comfortably held and restricted in terms of movement in a manner which seeks to minimise various potential sources of subject discomfort. Aspects may therefore be understood to have application in relation to obtaining an image of a substantially immobile human brain.

The body part support may be substantially or wholly formed from MRI compatible materials.

The term “MRI compatible” indicates that a material, component or device is safe for use in an MRI environment and/or can operate as intended in an MRI environment without generating significant MR signal artifacts. It will be appreciated that a material, component or device placed in a high-field strength region of magnetic field, may only be MRI compatible if it is suitably non-magnetic.

Magnetic fields induce magnetization in materials, the degree to which they do so varies. This is known as magnetic susceptibility.

It will be appreciated that a material, component or device placed in a strong magnetic field will interact with that field affecting the spatial distribution of the field. The stronger the interaction the more effect that material will have on the field and consequently on image quality that is dependent on the field distribution. Materials with differing magnetic susceptibilities placed close to one another will affect the magnetic field more than closely matched materials.

Materials comprising a biocompatible device would ideally closely match the magnetic susceptibility of the body part placed next to them. They may also differ but be placed in an arrangement to provide an improvement in magnetic field distribution.

The changing magnetic fields used in MRI scans can induce currents in conductive materials. These currents may adversely affect the image and a force may be exerted on the material.

The body part support may include one or more elements formed from a material selected to alter local magnetic field. The one or more elements may be locatable on or in the frame of the body part support. The one or more elements may comprise a passive shim. Accordingly, appropriate selection of material, dimension and/or location of the one or more elements may facilitate improved scan performance. In other words, scan performance can be improved by altering the local magnetic field by the use of materials and distribution with a particular magnetic susceptibility. According to some arrangements the one or more deformable pad, mountable on the frame may comprise such a passive shim. In some arrangements, the deformable pad may comprise appropriately doped material. In some arrangements, the deformable pad may comprise graphite doped foam material. The deformable pad comprising a passive shim may be provided on an interior surface of the frame and be dimensioned to substantially fill any gap between the body part and the frame. In arrangements where the body part to be supported comprises a head, the passive shim may be locatable in the region of a subject's forehead. In other such head support arrangements, one or more passive shim may be provided in the region of a subject ear or jaw. Provision of one or more passive shim can support creation of a more homogenous or predictable magnetic field within a body part to be imaged, and consequently support higher quality imaging of those body parts.

A second aspect provides a device including a body part support according to the first aspect.

The device may comprise an imaging apparatus.

The device may comprise an MRI coil.

The body part support may be integral to the device.

The body part support may be mountable within the device.

A third aspect provides a method of supporting a body part comprising: providing a body part support a frame and an inflatable bladder; the frame comprising a cover portion and a base portion, the cover and base portion being reversibly moveable with respect to each other between: an open position and a closed position; the method comprising: locating the body part within the base portion while the frame is in the open position; moving the frame to the closed position and securing the cover portion and base portion together to encircle at least a portion of an exterior surface of the body part located within the base portion; inflating the inflatable bladder mountable on the frame to cause a reduction of a gap formed between the interior of the frame and the exterior surface of the encircled portion of the body part located within the base portion.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function. Furthermore, embodiments of the invention described with respect to one aspect may be incorporated in a different aspect although not specifically described relative thereto.

### BRIEF DESCRIPTION OF THE DRAWINGS

Embodiments of the present invention will now be described further, with reference to the accompanying drawings, in which:

FIG. 1 is a perspective view of a head support in accordance with one arrangement;

FIG. 2 is a perspective view of the head support of FIG. 1, when in an open position;

FIG. 3a is a perspective view of some main components of a frame portion of the head support of FIG. 1;

FIG. 3b is an alternative perspective view of some main components of a frame portion of the head support of FIG. 1;

FIG. 4 is a perspective view of a manifold for use in conjunction with the head support of FIG. 1;

FIGS. 5a and 5b are schematic representations illustrating an inflation method for implementation in relation to the head support of FIG. 1;

FIG. 6 is a graphical comparison of head displacement in an MRI imaging scenario using (a) standard padding and (b) a head support as shown in FIG. 1;

FIG. 7 is a perspective view of a head support in accordance with one arrangement;

FIG. 8 is a perspective view of the head support of FIG. 7, when in an open position;

FIG. 9 is a perspective view of some main components of a frame portion of the head support of FIGS. 7 and 8;

FIG. 10 is a perspective view of a manifold for use in conjunction with the head support of FIG. 1 or FIG. 7.

FIG. 11 shows a range of pad positions and shapes for use in a head support as shown in FIG. 1 or FIG. 7

### DESCRIPTION OF THE EMBODIMENTS

Before describing one particular arrangement in detail, a general overview is provided. Arrangements described are suited to use with any appropriate scanning or imaging system, including X-ray, CT, ultrasound and MRI scanners. Whilst described in relation to imaging, it will be appreciated that a body part support or restraint described may have application outside imaging applications.

Arrangements generally relate to a body part support comprising: a frame and an inflatable bladder. The frame typically comprises two main portions, for example, a cover, cap or lid portion which can be placed over a base portion. The two main portions may be separable to allow easy insertion and removal of a body part to be supported or restrained. The two main portions may be open and closable with respect to each other between an open position in which a body part is locatable, without obstruction, within the base portion; and a closed position in which the cover portion and base portion are securable together such that they encircle at least a portion of the body part locatable within the base portion. The portion of the frame which encircles the body part to be supported or restrained provides a robust structure within which resistance to movement of the body part in various axes can be provided. The support includes at least one inflatable bladder. That inflatable bladder is mountable on an interior surface of the frame. The inflatable bladder can be located on the cover portion or base portion and is located in a position selected such that inflation of the inflatable bladder causes a reduction of a gap formed between the interior of the frame and encircled portion of the body part locatable within the base portion. In other words, inflation of the bladder causes a body part to become “trapped” between the frame and the inflatable bladder.

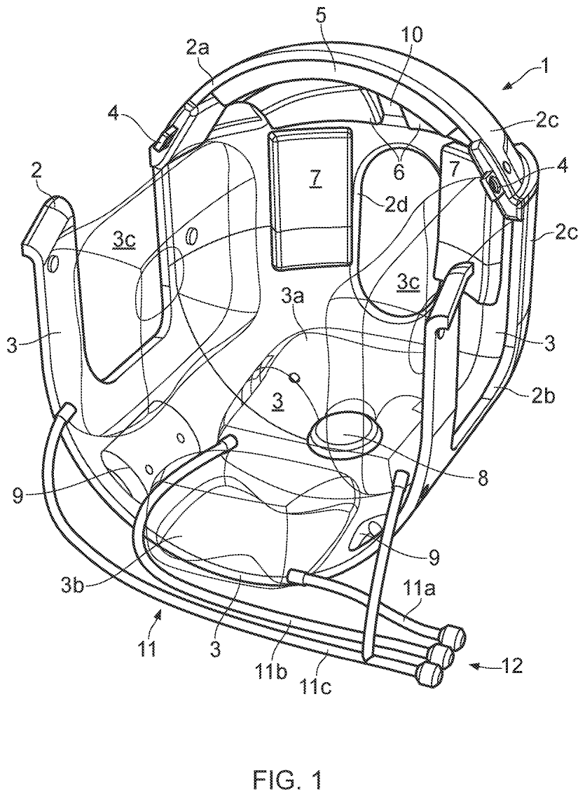

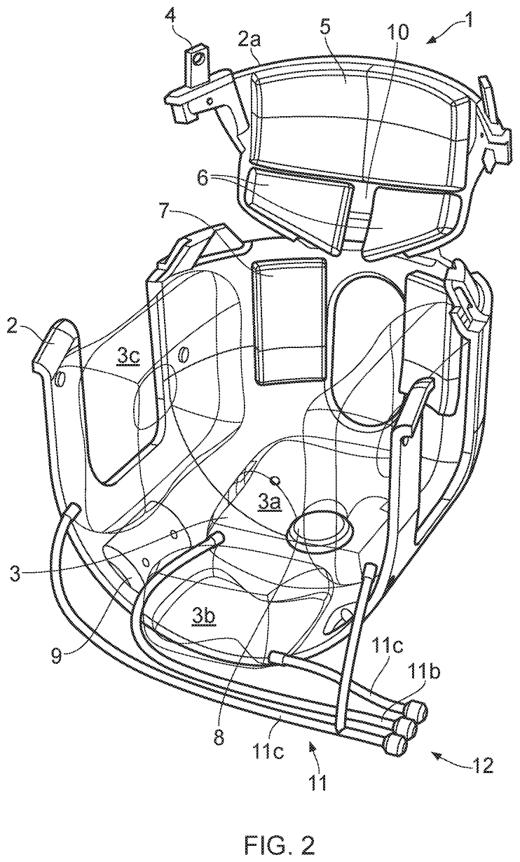

Turning now to one particular implementation, FIG. 1 is a perspective view of a head support 1 for use in an MRI imaging coil in accordance with one arrangement. The head support comprises: a frame 2 and a set of inflatable bladders 3. The frame of the head support comprises two main portions: a cap or lid portion 2a is located above a base portion 2b. The two main portions are hingeably connected. The closed position, in which the cap portion 2a is secured to the base portion 2b is shown in FIG. 1. The open position, in which the cap portion 2a is moved to allow easy insertion of a head into the base portion 2b, is shown and described in more detail in FIG. 2. The two main portions 2a and 2b of the frame 2 are openable and closable with respect to each other. In the closed position shown in FIG. 1, the cover portion 2a and base portion 2b are securable together by locking latches 4. The cover portion 2a and base portion 2b are locked in position with respect to each other to provide a section of the frame 2c which encircles a head placed in the support at around the temple. In the arrangement shown in FIG. 1, the cover and base portion together form a section of frame 2d which substantially encloses and surrounds the crown of a head within the head support.

The portion of the frame 2c which encircles the head provides a robust structure within which resistance to movement of the head in various axes can be provided. The support 1 includes two inflatable bladders 3a and 3b mounted on the base portion of the frame. The inflatable bladder 3a is located in the head support such that it sits beneath the occipital bone of the skull (external occipital protuberance or inion). The inflatable bladder 3b is located beneath the lower skull and neck, it is substantially wedge shaped and formed to accommodate the general shape of the neck and lower skull. Inflation of bladder 3a when a head is within the support lifts the head and causes a reduction of a gap formed between the interior of the frame 2c at the encircled portion of the head. Inflation of the wedge-shaped bladder 3b causes a rotation of the head up and back into the annular part of the frame 2c. The head in the support then becomes “held” between the frame 2c and the inflatable bladder 3a. To make that hold more comfortable to a subject, and to provide additional restraint and restriction to head movement, a foam pad 5 is provided on the part of the frame 2c substantially opposite the inflatable bladder 3a. In this instance, rather than provide a further inflatable bladder opposite bladder 3a, an arrangement has been implemented in which the weight of the head is supported by inflatable 3a and inflatable is connected to the base portion so that inflation of the bladder 3a causes the head to be pushed up into the appropriately positioned memory foam pad 5. In the arrangement shown, the memory foam pads used on the frame 2a opposing one of the inflatables conforms to the variations in the shape of the head and then does not further deform under the weight of the head. The pad has been located such that the weight of the head is not taken by those pads and instead is supported by the inflatable, the frame and the imaging device. By placing the pad such that gravity cannot load them, the tendency of such foam to progressively deform over time and with changing temperatures is mitigated. In the device shown in the Figures, a CONFOR foam pad 5 is provided on the upper inside face of the ‘halo’ part of the frame. CONFOR foam is an aviation grade material developed for its slow rebound properties and comes in several grades. An appropriate grade can be selected in dependence upon the body part typically to be placed within a body part support.

The head support 1 shown in FIG. 1 also includes a pair of inflatable bladders 3c. The inflatable bladders 3c are locatable diametrically opposite each other on the interior of the frame 2. The bladders 3c are provided to support or restrain the head against lateral movement. As shown in FIG. 1, the pair of inflatable bladders are provided either side of the inflatable bladders 3a, 3b. Inflation of the pair of bladders 3c causes a reduction of a gap formed between the bladders 3c and the head located in the support. The inflatable bladders 3c are shaped, by way of structuring the interior of the bladders 3c. Such shaping, in the form of divots to accommodate ears, allows for restraint of the head without crushing the ears and also for sealing against the head to attenuate airborne sound, these bladders also provision for audio attenuating materials near the ear, which can help a subject feel more comfortable, and therefore less likely to move, in a loud MRI scanner bore. The inflatable 3c bladders include accommodation for audio delivery (for example, via ear buds) while maintaining an attenuating ‘seal’ around the outer ear. A canal is included in an alternative design to allow unrestricted passage of the connecting cables/tubes to the audio delivery device.

In the arrangement shown in FIG. 1, the pair of inflatables 3c are attached to the frame 2b by elastic. Use of elastic connectors allows for variation in subject ear position. The inflatables 3a and 3b use a hinging mechanism in relation to the frame 2. Inflating the bladders with one end anchored creates a turning moment (torque). This causes a rotation of the bladders relative to the frame 2 allowing for orientation of a head within the support 1 in a controlled manner. The inflatable bladder 3b is attached to the frame 2b mechanically at one end by shaped clamp plates 9. The inflatable bladder 3a is attached to the frame 2 mechanically by screws near the crown section 2d. The shaped clamp plates 9 may further enable provision for monitoring sensors, cooling air delivery, stimulation delivery (for fMRI studies) and further padding supports as required.

The head support shown in FIG. 1 further comprises foam pads 6, provided in the top of the frame 2a behind the foam pad 5 and pads 7 provided in the crown of the frame 2d and pad 8 provided beneath the inflatable bladder 3a. Pads 6 and 7, taken together with the wedge-shaped inflatable 3b, and the shape of the head, act together to further restrict movement of the head in relation to the frame 2. Pad 8 provides comfort for the external occipital protuberance (inion) while the participant is loaded and prior to inflation of the inflatable bladder 3a.

The various pads enable an MRI operator to alter the alignment of a head while a subject is located within the restricted space of an MRI coil.

FIG. 2 is a perspective view of the head support of FIG. 1, when in an open position. The frame in the example shown has a cover portion 2a which is hingeably connected by a hinge 10 to the base portion 2b and allows for a front half of the head support 1 to be opened. Such an arrangement allows easy ingress and egress of a head into the support 1. The hinge and lock mechanisms are arranged to be low profile and do not protrude into the volume where the body part is to be located. As shown in the Figures, a slim latching mechanism and hinge are used in this implementation.

The closure of the frame sections 2a and 2b create the annular or “encircling” section 2c. Once in a closed configuration the frame restricts exit of the participant from the support 1. The latches 4 are designed with integrated connection points for a ‘release cord’ (not shown). Pulling on this cord by either the MRI operator or the participant will release the frame section 2a allowing it to open and release the participant from the support 1. Since the support 1 is not affixed to the MRI headcoil, the participant may be unloaded from the scanner without any restriction in the event of an emergency.

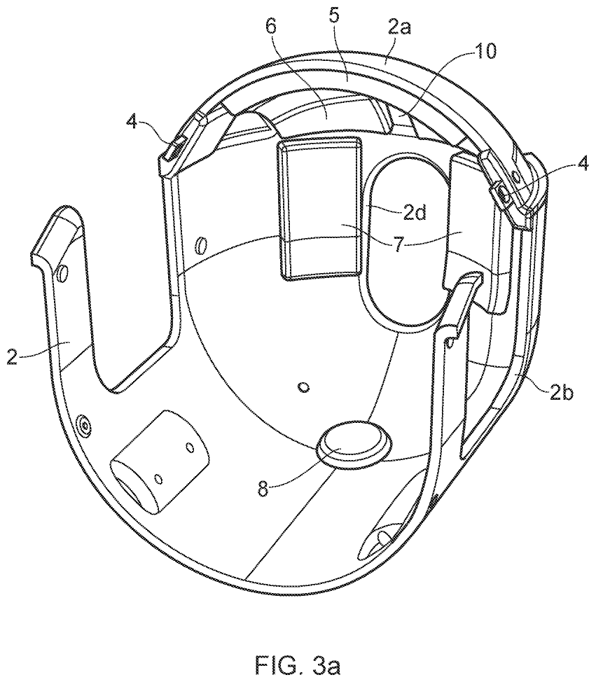

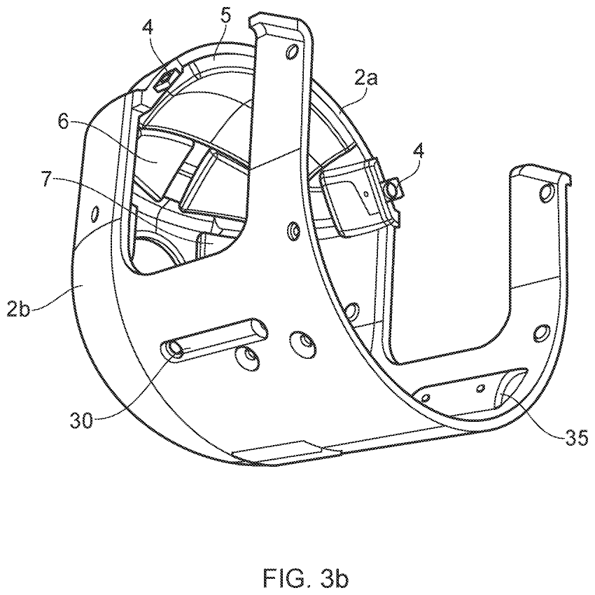

FIG. 3a is a perspective view of some main components of a frame portion of the head support of FIG. 1, in which the inflatable bladders 3 have been removed for the purposes of clarity. FIG. 3b is an alternative perspective view of some main components of a frame portion of the head support of FIG. 1, in which some features of the external surface of the frame may be seen in more detail. The frame shown in FIG. 3b includes a pair of cooling passages 30 in the form of channels or conduits provided on the exterior surface of the frame in the region of the ears. The cooling passages form part of a cooling circuit and connector blocks 35 are provided on the interior surface of the frame for connection of the passages to a source of cooling air flow. As shown in FIG. 3b, the cooling passages 30 comprise open topped trenches provided in an exterior surface of the frame. Accordingly, the open channel or trench may facilitate easy cleaning. Although only two cooling passages 30 are shown in FIGS. 3a and 3b, it will be appreciated that a frame may be formed such that cooling passages, channels or conduits may be located on the support frame in one or more positions to cool a body part locatable within the support.

The head support of FIGS. 1 to 3 includes inflatables and pads placed within the frame 2 such that they are spaced apart from each other to allow free airflow paths around a head within the support. The pads and inflatables are at least partly discrete in support of such separation. Maintenance of airflow can aid maintenance of a comfortable temperature and humidity and/or assist in provision of head cooling in the restricted and sometimes warm environment within an MRI coil.

The exterior surface of the head support 1 is shaped to conform to the interior of the MRI coil within which it is to be placed. The support 1 has exterior dimensions selected to conform and grip to the MRI head coil.

### Connections, Air Lines and Manifold

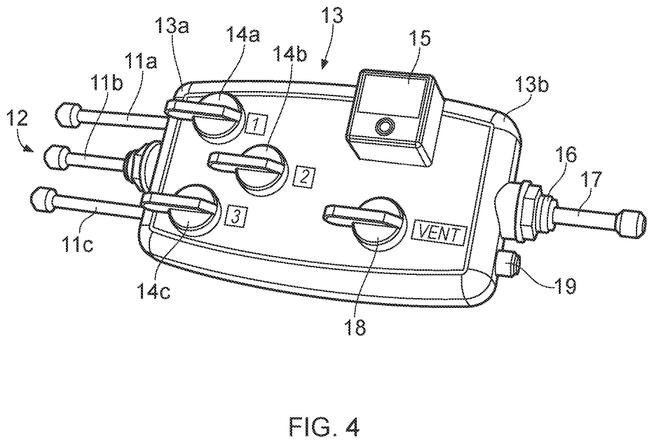

FIG. 1 shows the inflatable bladders 3a, 3b and 3c connected via 11a,11b and 11c respectively to an air management manifold shown in more detail in FIG. 4. The connection of bladders 3a, 3b, 3c is implemented in the arrangement shown via quick release connectors 12. In the arrangement shown in FIGS. 1 and 2, the two inflatable bladders 3c are interconnected so that the participants head is exposed to substantially balanced forces from the pair of inflatables and the head position is centralised within the support upon inflation of the pair of inflatable bladders 3c.

FIG. 4 is a perspective view of a manifold 13 for use in conjunction with the head support of FIG. 1. The manifold can typically be positioned next to a participant on an MRI scanner bed. The manifold will typically be operated by the MRI operator while the participant is loaded. It will be appreciated that in some implementations, a participant may be able to operate the manifold themselves with appropriate instruction. Supply hoses 11 of the manifold are connectable to the inflatable bladders 3 shown in FIG. 1. Each of the supply hoses 11 are coupleable to an appropriate port on the manifold 13a. Each of the ports can be opened or closed by one of three ball valves 14a, 14b and 14c shown labelled as [1], [2] and [3] respectively to indicate the usual order of inflation. The rear face of the manifold 13b includes a pump port 16 coupleable, via a hose 17, to a hand operated inflation bulb as found on a blood pressure inflatable device (not shown). The bulb includes an integrated ball valve enabling fine adjustment of the pressure by the operator. During inflation, an indication of pressure in the current open hose(s) 11a, 11b, 11c is displayed on an illuminated digital pressure meter 15 mounted to an upper surface of the manifold 13. A vent valve 18 allows release of pressure by an MRI operator for participant loading and unloading and in the case of emergency. Opening valves 14 and the vent 18 returns the inflatables 3 to atmospheric pressure. A one-way pressure relief valve 19 is set to vent at a predetermined maximum pressure level (selected in dependence upon body part type to be supported) in order to protect both a participant and the inflatable bladders 3 from exposure to excessive pressure.

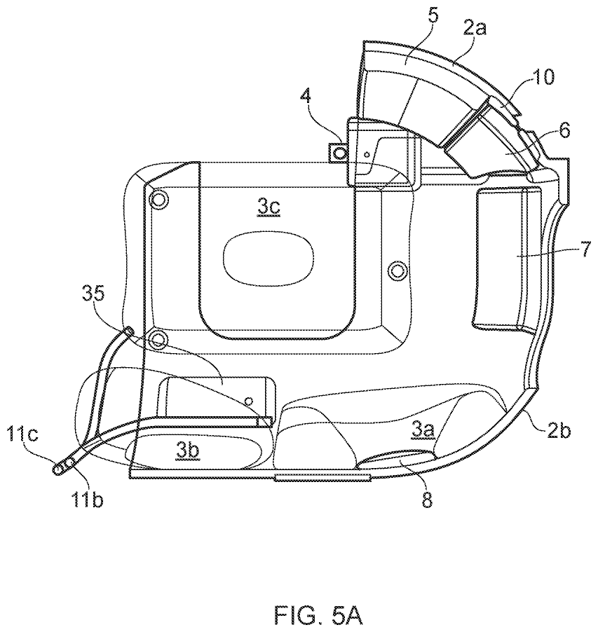

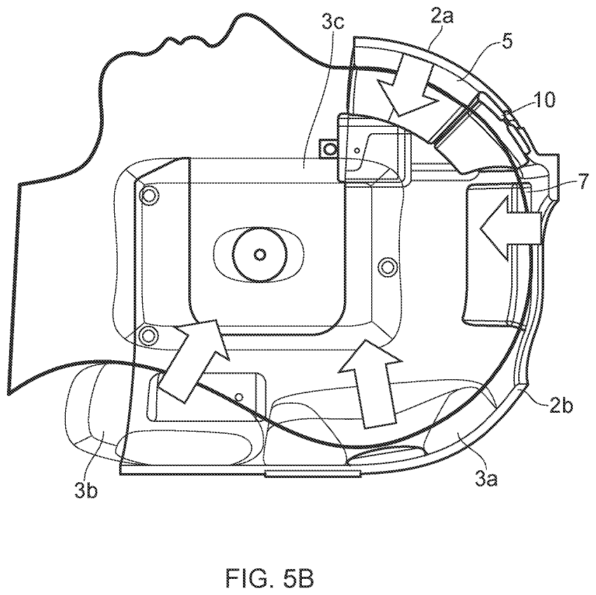

FIGS. 5a and 5b are schematic representations illustrating an inflation method for implementation in relation to the head support of FIG. 1. FIG. 5a is a cross-sectional view of the support. FIG. 5b is a cross-sectional view of the support including a representation of the head of a participant after inflation of all inflatable bladders. Illustrative arrows are provided in FIGS. 5a and 5b showing a general direction of a force exertable by the pads (black arrows) and bladders (white arrows) upon a head locatable (as shown in FIG. 5b) within the head support.

In general, a typical participant loading procedure comprises the following steps:
The participant lays back, placing their head into the support 1, whilst the frame is in the open position.
The frame 2a of the support is moved to a closed position and locked, using latches 4 into position. The locking mechanism secures the cover portion 2a to the lower portion 2b of the frame.
An MRI operator may arrange the uninflated pair of inflatables bladders 3c generally over the ears of the participant. Visual placement is aided if translucent material is used to form the pair of inflatable bladders 3c.
The MRI operator inflates the inflatable bladders 3a, 3b and 3c sequentially. Each bladder is inflated to a preselected or desired pressure. That pressure may differ between inflatable bladders. The pressure(s) may be selected in dependence upon anticipated participant head dimensions (for example, adult/child/male/female/Caucasian and similar characteristics). The predetermined pressure is selected to comfortably and securely hold the head of a participant within the frame.
The manifold is placed on the scanner bed next to the patient and normal MRI loading and scanning commences.
For egress from a manifold, such as the example shown in FIG. 4, is used to sequentially vent the bladders 3 to atmospheric pressure, the latches 4a are released and the frame 2 opened. Once the frame is opened, a participant may be free to remove their head from the support and sit up.

In the example shown in detail, all of the components shown in the support 1 of FIGS. 1 to 3 and the manifold of FIG. 4 are formed from MRI compatible materials, and so can be placed and operated if wholly within an operational MRI scanner. The term “MRI compatible” indicates that a material, component or device is safe for use in an MRI environment and/or can operate as intended in an MRI environment without generating significant MR signal artifacts. In alternative implementations, the body part support may include one or more elements formed from a material selected to alter local magnetic field. The one or more elements may be locatable on or in the frame of the body part support. The one or more elements may comprise a passive shim. Accordingly, appropriate selection of material, dimension and/or location of the one or more elements may facilitate improved scan performance. In other words, scan performance can be improved by inclusion in the support of an element or shim which alters the local magnetic field by the use of materials and distribution with a particular magnetic susceptibility.

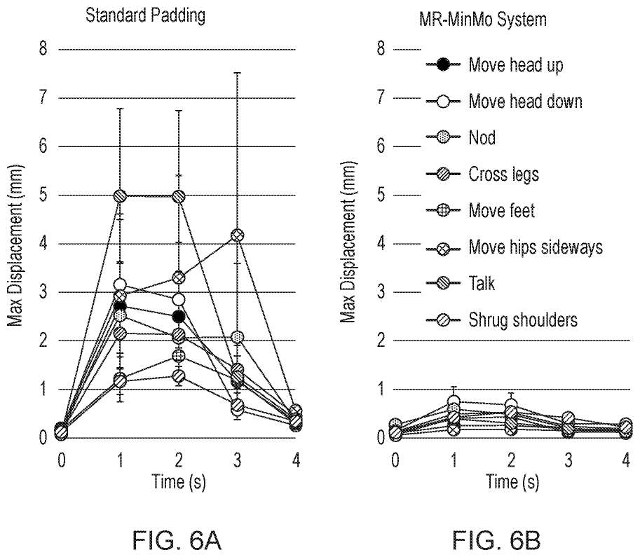

FIG. 6 is a graphical comparison of head displacement in an MRI imaging scenario using (a) standard padding and (b) a head support as shown in FIG. 1. It can be seen that the head support shown in FIGS. 1 to 3 can reduce maximum head displacement roughly five-fold compared to use of standard padding. 3 patients performed transient body and head movements characteristic of patients during fast EPI scanning.

An alternative implementation in accordance with arrangements is now described in more detail. Like features of the alternative implementation are accorded like reference numerals to the implementations described in relation to FIGS. 1 to 6.

It will be appreciated that implementations of arrangements seek to stabilise and support a body part of a participant and prevent as much motion as possible. In the case of an MRI head support, implementations seek to support a participant head in a repeatable position relative to the coil and protect the coil from any forces induced by the body part support.

Implementations suited to imaging applications may be configured or arranged to enable swift extraction of a participant from the body part support and/or imaging apparatus into which the body part support is placed, for example, an MRI coil.

Implementations of arrangements may be dimensioned, shaped or otherwise configured to accommodate a range of body part dimensions in a manner which is comfortable to a participant using the body part support. For example, in an MRI head support application, features of the body part support may be selected, dimensioned or arranged to conform to a large range of typical head sizes and shapes.

A body part support in accordance with implementations of arrangements may be operable to allow a simple or standardised participant loading procedure.

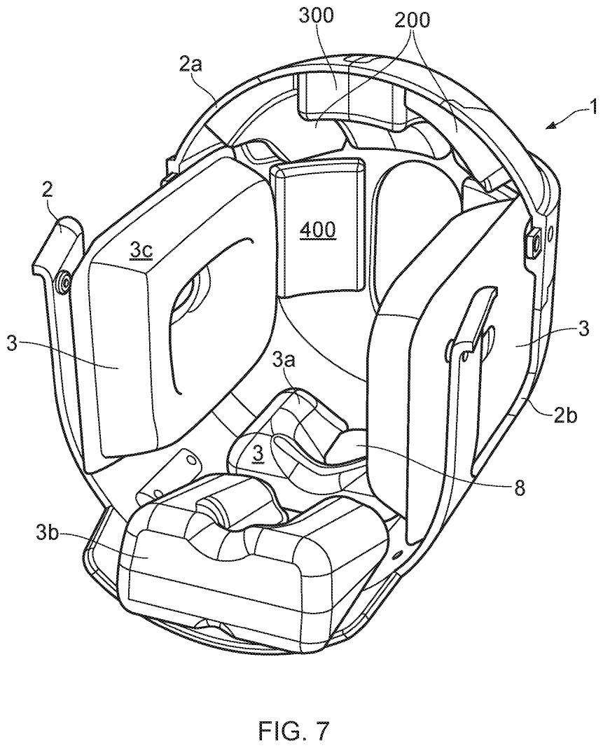

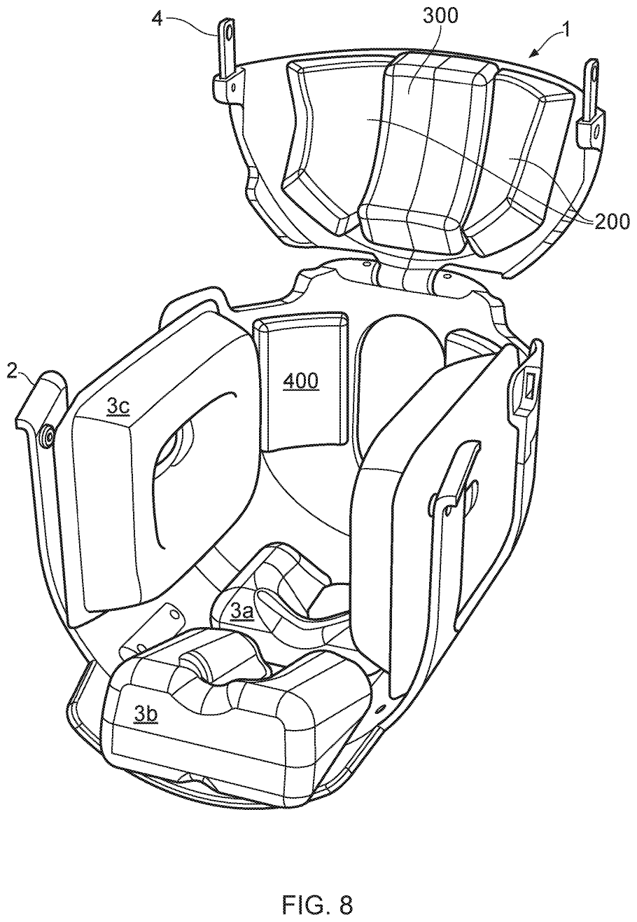

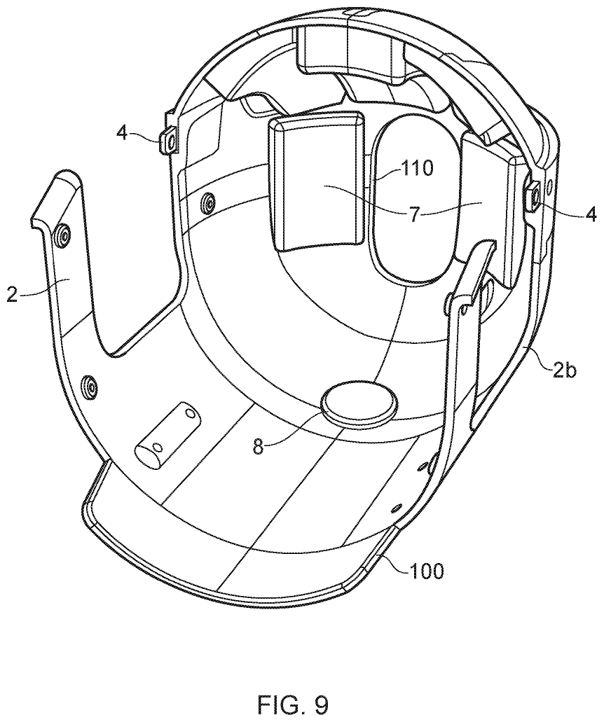

FIGS. 7 to 9 are schematic representations of an alternative head support in accordance with arrangements. In particular, FIG. 7 is a perspective view of a head support in accordance with one arrangement; FIG. 8 is a perspective view of the head support of FIG. 7, when in an open position; and FIG. 9 is a perspective view of some main components of a frame portion of the head support of FIGS. 7 and 8.

### Frame

As shown in FIG. 9, according to one implementation of a head support for MRI imaging applications, a frame is provided. FIG. 9 is a perspective view of some main components of a frame portion of the head support of FIG. 7, in which the inflatable bladders 3 have been removed for the purposes of clarity. The head support of FIGS. 7 to 9 includes inflatables and pads placed within the frame 2 such that they are spaced apart from each other to allow free airflow paths around a head within the support. The pads and inflatables are at least partly discrete in support of such separation and airflow. Maintenance of airflow can aid maintenance of a comfortable temperature and humidity and/or assist in provision of head cooling in the restricted and sometimes warm environment within an MRI coil.

The exterior surface of the head support 1 of FIGS. 7 to 9 is shaped to conform to the interior of an MRI coil within which it is to be placed. In particular, the support 1 has exterior dimensions selected to conform and grip to the MRI head coil. The implementation of FIGS. 7 to 9 comprises a frame 2 which comprises a 3D printed shell of stiff plastic having exterior dimensions designed to closely follow contours of an imaging apparatus, for example, MRI head-coil, into which the body part support is to be placed. The stiff frame 2 provides mounting points for the various body part support stability modules and features in the form of inflatables and pads described further below and which locate a body part within the support. The frame 2 operates to protect the imaging apparatus, for example, MRI coil, from damage and can be further configured to locate components of a fluid delivery system to inflate the inflatable bladders 3.

The components of the frame portions, 2a and 2b are shaped such that when in a closed position, an interior surface of the frame 2 is substantially smooth. A smooth interior surface of the body part support may increase participant comfort, and may avoid creation of potential pressure points when in a closed position encircling a body part.

The frame 2 of the support shown in FIGS. 7 to 9 includes cutaway sections in the lower portion 2b of the frame to allow space for an outer ear of a participant and visibility for ear-pad positioning. The form of the cut away sections was determined by examining the Liverpool-York Head Model (LYHM) database for an indication of average ear position and dimension and through volunteer testing. In general, such features of a body part support which are selected for user comfort can be determined based on a population average ideal model of a given body part.

As can be seen in FIG. 9, the head support frame includes an extended neck panel 100, integrally formed with the frame 2. That extended neck support panel 100 provides a mechanism to support neck inflatable bladder(s) outside the main head support. In particular, in relation to a head support, locating the inflatable bladder (not shown in FIG. 9) on the extended neck support allows the inflatable to be located at a position in which the inflatable aids correct positioning of the body part (head) inside the frame. In the implementation shown in FIGS. 7 to 9, the extended neck support moves the location of a head rotation axis (nodding) to provide comfortable and reliable head stabilisation within the support 1.

The head support of FIGS. 7 to 9 includes a cut out portion 110. That cut out in the bottom portion of the frame 2b allows a head of a participant to be positioned as far superior in the head support as possible. The location and dimension of the cut out portion 110 is selected to accommodate average user skull shape. In the example shown in FIGS. 7 to 9 that location and dimension is informed by the LYHM.

The head support of FIGS. 7 to 9 comprises a hinged halo portion which encircles at least a portion of an exterior surface of the body part (head) locatable within the body part support. The halo portion is formed between the upper part of the frame 2a and the base portion of the frame 2b. The location of hinge 10, linking the upper and lower parts of the frame 2, is selected so that the rotational axis maximises a ‘skin tautening’ force applied to a user forehead when the frame is in the closed position. By tautening skin on a body part as it is held within a body part support, it is possible to limit movement of the body part within the support.

The frame shown in FIGS. 7 to 9 comprises mounting points for one or more inflatable bladder and/or support pad in specific locations selected for appropriate support and positioning of a body part within the support.

### Inflatables

FIGS. 7 and 8 show an implementation of a head support including inflatables and pads which differ from the inflatables and pads of the head support shown in FIGS. 1 to 3. FIGS. 7 and 8 show a head support including a set of modular polyurethane inflatables which were iteratively designed to anatomically accommodate a typical participant neck, ears and inion (protrusion at the rear of the skull, part of the occipital bone).

Each of the inflatable bladders which form part of the head support shown in FIGS. 7 and 8 has various specifically designed features based on the support it is desired to provide to the body part (head) locatable within the frame.

### Neck Inflatable (3b)

The neck inflatable 3b shown in FIGS. 7 and 8 comprises a bifurcated bladder. Provision of a bifurcated inflatable allows support to be provided to the body part (in this instance, head) in a manner which reduces undesirable pressure or force on a protrusion or more rigid region of a body part to be supported. In this instance, the bifurcated inflatable reduces pressure on the spine.

The neck inflatable 3b of FIGS. 7 and 8 is dimensioned such that it is larger at the inferior end and tapers down, matching the average curve of the neck at the point the inflatable is mounted on the frame 2. The mounting point of the neck inflatable in the implementation shown in FIGS. 7 and 8 is located at the superior end of the inflatable (within the frame) so that as the neck bladder is inflated it acts to pivot the head of a user up and into the support. In other words, the neck support is shaped and attached to the frame such that inflation of the neck bladder pushes the head ‘up’ into the body part support.

### Inion Inflatable (3a)

The inion inflatable shown in FIGS. 1 to 3 has a torus shape, and is mounted on a flat surface. The inion inflatable shown in FIGS. 7 and 8 comprises a C-shaped bladder, which may be preferred by users by comparison to a full ring. The C-shaped inflatable bladder shown in FIGS. 7 and 8 is mountable at the ends of the ‘c’ such that, as inflation occurs, the body of the bladder is configured to push the body part within the support pivot up and into the frame 2. In the case of the head support shown in FIGS. 7 and 8, the C-shaped bladder pushes a participant head up and back into the frame and lifts a participant's forehead into the forehead pads (described below) mounted on the portion of the frame which encircles the head.

### Ear Inflatables (3c)

The ear inflatables 3c shown in FIGS. 7 and 8 are slightly tapered square-shaped tori, one for either side of the head. They are mounted on elastic fixings to allow for a variation in user ear position and to accommodate sound delivery to a user located in the frame via, for example, earbuds. Once inflated the ear inflatables grip firmly against a textured inside surface of the frame 2, and balance a participant's head within the centre of the support. The taper of the ear inflatables encourages the head upwards into the frame. The ear inflatables are made from a clear or translucent PU film to allow careful and repeatable comfortable positioning over an outer ear portion of a participant. Avoiding undesirable pressure on delicate regions of a body part to be supported can help ensure participant comfort.

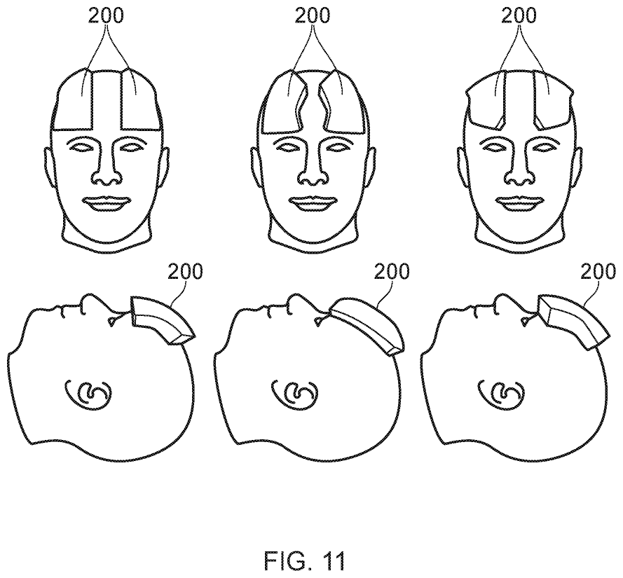

FIG. 11 shows a range of pad positions and shapes for use in a head support as shown in FIG. 1 or FIG. 7

### Pads

As described in relation to the head support shown in FIGS. 1 to 3, a body part support may comprise one or more pads which are used in conjunction with the inflatable bladders to support a body part in position within a frame.

### Forehead Pads

In the implementation of a head support shown in FIGS. 7 and 8, forehead padding is provided in the form of two or more pads, or pad portions having different structural and deformability characteristics. A single pad, as shown in FIGS. 1 to 3 may create pressure points since a single pad may not easily accommodate a range of head shapes. More generally, to accommodate a range of body part dimensions, it may be possible to provide padding having different material characteristics to better accommodate a range of body part dimensions.

### Forehead: Split ‘Horn’ Pads

FIG. 11 illustrates schematically a pair of pads 200 which can be provided on the upper portion of a frame 2a of a head support such as that shown in FIG. 1 or FIG. 7. As shown in FIG. 11, the forehead padding may comprise a pair of mirrored pads 200. Providing a gap between the pair of pads may reduce pressure exerted by the body part support 1 upon the most sensitive areas of the forehead. The most sensitive part(s) of a body part may be determined as reported from participant testing and/or via nerve anatomy investigation.

The two pads shown in FIG. 11 have an inner surface specifically curved to match the first two principle modes of variation averages from the LYHM for males and females (combined). As shown in FIG. 11, the pads can be mounted on the upper part of the frame 2a in various orientations. The first column of FIG. 11 shows the most commonly used orientation, and the separation of the pads can be adjusted to accommodate more, or less, curved foreheads. The layout shown in column two of FIG. 11 better conforms to SE Asian diaspora head shapes (more rounded, flatter back and forehead) whilst the arrangement shown in column three of FIG. 11 is more effective for flatter foreheads: those that fall away less sharply, typically children and women. All of the pads of FIG. 11 have the same general mirrored pair shape, and their orientation can be adjusted as shown to accommodate a range of head shapes.

These shaping of the pads 200 and their mounting position on the frame help to, when combined with the specifically chosen pivot point of the hinge 10 between the lower 2a and upper 2a portions of the frame, induce a tautening of user skin. The tautening occurs as the pads and inflatable bladders, and frame itself, work together to apply a force upon a user head up and back away from user eyebrows. That skin tautening can greatly increases resistance to a user nodding and moving within the support, but does not induce excessive discomfort.

### Forehead: ‘Ang’ Pad

As shown in FIGS. 7 and 8, in the implementation of a head support shown, a thick and soft memory foam block 300 is mounted in the gap between pads 200. Pad 300 accommodates a user head shape largely irrespective of head dimension and is significantly softer and more resilient than the material selected for pads 200. Provision of comfort “Ang” pad 300 functions to provides an increased surface area for skin tension and pressure point reduction functions.

### Head: ‘Di-Hawk’ Pads

As shown in FIGS. 7 and 8, in the implementation of a head support shown, a pair of thin memory foam pads 400 are mounted on a top inner surface of the base portion 2b of the frame. These pads 400, in use, make contact with the top of a participant's head and can help to reduce user nodding (pitch) motion, particularly if used in conjunction with the pads 200 and 300 referred to above.

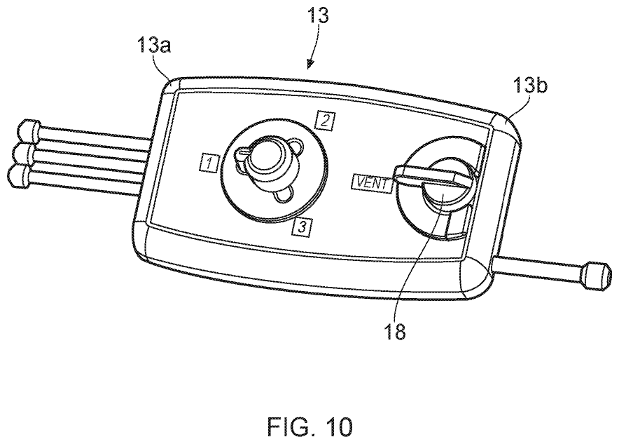

FIG. 10 is a perspective view of a manifold for use in conjunction with the head support of FIG. 1 or FIG. 7.

### Manifold

As described in relation to FIGS. 1 to 4, fluid delivery to the inflatable bladders 3 can be controlled by a fluid delivery manifold 13. In some implementations, in addition to a main fill and release function, air can be pumped to the manifold by a hand pump bulb with attached vent valve, thereby supporting fine tuning of bladder inflation by an operator. The manifold shown in FIG. 10 comprises a single input control selector 14 which allows a user to engage appropriate valves in support of inflation of each of the inflatable types (3a, 3b, 3c).

The manifold system shown in FIG. 10 further comprises an automatic pressure relief valve (not shown) to prevent damage to the frame/coil/inflatables and to maintain participant comfort. The manifold shown in FIG. 10 comprises a vent valve 18 for deflation of all inflatables. Provision of such a rapid release valve allow swift egress of a body part from a body part support in emergencies. The manifold of FIG. 10 is connectable to a selectable vacuum system, use of which may allow for rapid deflation of one or more inflatable bladder 3 before participant loading, thus allowing more space and ease of entry to the body part support.

The manifold of FIGS. 4 and 10 are designed such that they are dimensioned to be locatable within restricted spaces. For example, in relation to the head supports shown in FIG. 1 and FIG. 7, the manifold can be placed to the side of the head-coil during

MRI scanning of a participant. In such an implementation, all components of the manifold may be selected to be formed from a material which is MRI compatible. In particular, for MRI applications, the components of the body part support and manifold may be chosen such that they do not contain metal or MR-visible materials.

### Peripherals

### Hairnets

In use, a head support such as that shown in FIGS. 1 and 7 may be best used by a participant in various ways. In one implementation, use of a hairnet is suggested. Use of a hairnet may prevent stray hair catching in the various mechanisms of the head support. A hairnet may control and compress a range of typical hairstyles found in the population. Furthermore, a hairnet may aid the efficacy of skin tautening applied by a head support to a head located within the support, thereby limiting user movement within the support. A hairnet can distribute tautening forces applied by the various pads and inflatables to a user and therefore act to increase user comfort. It will be appreciated that use of a hairnet can serve a hygiene role by creating a physical barrier between a user and an inner surface of the head support.

### Use of a Head Support—User Loading

Various ways to load a user into a head support such as that shown in FIGS. 1 and 7 are possible. One possible workflow is described below:

Before MRI scanning, a head support device such as that shown in FIG. 1 or FIG. 7 can be placed in an MRI scanner head coil. The neck cushion bladder 3b and inion 3a bladder may be semi-inflated, and the top frame portion 2a (halo) rotated back around hinge 10 to an open position. The open position allows a user head to be loaded into the head support. As part of one possible workflow, ear cushion bladders 3c may be deflated.

A participant may then be seated on an MRI scanner gurney with knees bent, and a hair net placed over the participant's head.

An MRI operator guides the participant's head into the frame, making sure that the crown of the head contacts the cushioned pads (for example, pads 400, provided in the base of the frame 2b), and that the head is slightly rotated with chin up. A ‘chin up’ posture is encouraged before closing the ‘halo’, this creates the required skin tension when the halo is closed and the chin relaxes to a normal attitude. The operator slowly closes the halo portion of the frame 2a, such that all cushions 200, 300 located on that frame portion are in contact with the participant's forehead and temples.

The top of the MRI head coil can then be placed over the halo 2a. The operator may then consecutively inflate: the neck 3b, inion 3a, and ear 3c inflatable bladders, ensuring that the pressure is sufficient to secure the head within the head support but not too great so as to induce discomfort in the participant. The operator may also ensure that the ear cushions 3c are located appropriately over the ears of a participant.

Earbuds can be inserted by a participant before lying the participant back into the head support. In some implementations, channels in the ear inflatables 3c may be incorporated for larger diameter audio delivery tubing.

An under-knee bolster cushion, commonly found in MRI suites, is useful in preventing the participant slipping foot-wise since this is the least restrained direction of translation in the head support device shown in FIGS. 1 and 7.

Although described in relation to a head support for MRI applications, it will be appreciated that a body part support operating in accordance with the general principles described herein may be used across various applications. In terms of medical imaging applications, a body part support in accordance with the principles described herein may be useful in Magnetoencephalography (MEG), Positron emission tomography (PET), PET-MRI, CT-PET and (optically-pumped-magnetometer) OPM imaging.

Arrangements are described more fully with reference to the accompanying drawings, in which some embodiments are shown. Arrangements can be embodied in many different forms and should not be construed as limited to the particular illustrated embodiments described in detail. The detailed description of some embodiments is provided so that this disclosure is sufficient to convey the underlying principles to those skilled in the art.

Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

1. A body part support comprising:
a frame and an inflatable bladder; wherein:
the frame comprises a cover portion and a base portion, the cover portion and base portion being reversibly moveable with respect to each other between:
an open position in which a body part is locatable within the base portion; and
a closed position in which the cover portion and base portion are securable together to encircle at leasta portion of an exterior surface of the body part locatable within the base portion;
the inflatable bladder being mountable on the frame, located such that inflation of the inflatable bladder causes a reduction of a gap formed between an interior of the frame and the encircled portion of the exterior surface of the body part locatable within the base portion.

2. The body part support according to claim 1, wherein the frame is formed from a material having a rigidity sufficient to resist significant deformation when the inflatable bladder is fully inflated and the body part locatable within the base portion is in position.

3. The body part support according to claim 1, wherein external surfaces of the body part support are shaped to conform to an interior surface of an imaging device within which the body part support is locatable.

4. The body part support according to claim 1, wherein the cover portion and base portion are hingeably connected, wherein the hingeable connection between the cover portion and base portion is located such that, in the closed position, at least one of the cover portion or base portion is configured to exert force transverse to the exterior surface of the body part locatable within the body part support.

5. The body part support according to claim 1, wherein the body part support comprises one or more cooperating elements configured to lock the cover portion and base portion in the closed position.

6. The body part support according to claim 1, wherein the inflatable bladder is formed from a pliant material.

7. The body part support according to claim 1, wherein the inflatable bladder inflates to form a wedge.

8. The body part support according to claim 1, wherein the inflatable bladder comprises an opening configured to receive fluid from a fluid source, the opening further configured to allow exit of gas from the inflatable bladder.

9. (canceled)

10. The body part support according to claim 1, wherein the body part support comprises a deformable pad, mountable on an interior surface of the frame facing the inflatable bladder.

11. The body part support according to claim 1, wherein the body part support comprises a pair of inflatable bladders locatable facing each other on the interior of the frame, and wherein inflation of the pair of bladders causes a reduction of a gap formed between the pair of inflatable bladders and the body part locatable within the base portion.

12. (canceled)

13. The body part support according to claim 11, wherein the pair of inflatable bladders are formed from a pliant material.

14. The body part support according to claim 11, wherein each of the pair of inflatable bladders is independently inflatable.

15. The body part support according to claim 11, wherein each of the pair of inflatable bladders comprises an opening configured to receive gas from a gas source.

16. (canceled)

17. The body part support according to claim 1, wherein the inflatable bladder mountable on the frame comprises one or more cells in fluid communication, the one or more cells being dimensioned to give the inflatable bladder a predetermined shape when inflated.

18. (canceled)

19. The body part support according to claim 1, wherein the body part support comprises a fluid flow control manifold, coupleable to: (i) an inflatable bladder opening configured to receive fluid from a fluid source, and (ii) a fluid source.

20. The body part support according to claim 19, wherein the fluid flow control manifold comprises one or more valves reconfigurable to allow or prevent fluid flow from a fluid source to the inflatable bladder.

21. The body part support according to claim 19, wherein the fluid flow control manifold comprises a pressure meter.

22. (canceled)

23. (canceled)

24. (canceled)

25. The body part support according to claim 1, wherein the body part support comprises a pressure sensor associated with the inflatable bladder.

26. The body part support according to claim 1, wherein the body part support comprises at least one deformable pad slidably mountable on the frame.

27. The body part support according to claim 26, wherein the inflatable bladder and at least one deformable pad are dimensioned to be mountable at positions on the frame in which there is a space between the inflatable bladder and the at least one deformable pad.

28. The body part support according to claim 1, wherein the body part support comprises a head support.

29. The body part support according to claim 1, wherein the body part support comprises an MRI imaging head support.

30. The body part support according to claim 1, wherein the body part support is wholly formed from MRI compatible materials.

31. (canceled)

32. (canceled)

33. (canceled)

34. (canceled)

35. (canceled)

36. A method of supporting a body part comprising:
providing a body part support, a frame, and an inflatable bladder, the frame comprising a cover portion and a base portion, the cover portion and base portion being reversibly moveable with respect to each other between an open position and a closed position;
locating the body part within the base portion while the frame is in the open position;
moving the frame to the closed position and securing the cover portion and base portion together to encircle at least a portion of an exterior surface of the body part located within the base portion;
inflating the inflatable bladder mountable on the frame to cause a reduction of a gap formed between an interior of the frame and the encircled portion of the exterior surface of the body part located within the base portion.

37. (canceled)

38. The body part support according to claim 1, wherein the inflatable bladder comprises a bifurcated bladder which, when inflated, is configured to centre a body part locatable within the body part support.

39. The body part support according to claim 1, wherein the inflatable bladder is attachable to the frame at a position selected such that inflation of the inflatable bladder causes the inflatable bladder to move the body part locatable within the base portion such that the exterior surface of the body part opposing an interior surface of the frame opposite the inflatable bladder is moved in a direction having a component transverse to the interior surface of the frame.

40. (canceled)
